(19) United States
(12) Plant Patent Application Publication
(10) Pub. No.: US 2008/0196132 P1
(43) Pub. Date: Aug. 14, 2008

(54) American elm tree named 'St. Croix'

(76) Inventors: Christian Bliska, Afton, MN (US); Patricia Bliska, Afton, MN (US); Mark Stennes, New Brighton, MN (US); Chad Giblin, Minneapolis, MN (US)

Correspondence Address: BARNES & THORNBURG LLP, P.O. BOX 2786, CHICAGO, IL, 60690-2786, US

(21) Appl. No.: 11/704,772

(22) Filed: Feb. 9, 2007

(51) Int. Cl. A01H 5/00

(52) U.S. Cl. PLT/221

(57) ABSTRACT

A new and distinct variety of American elm tree, particularly distinguished by substantial tolerance to an epiphytotic and normally deadly vascular wilt disease of the genus Ulmus known as Dutch elm disease.

### LATIN NAME

Ulmus americana L.

### VARIETAL DENOMINATION

‘St. Croix’

### FIELD OF THE INVENTION

This invention relates to a new and distinct variety or cultivar of the deciduous tree Ulmus americana, commonly known as the American elm.

### BACKGROUND OF THE INVENTION

This new variety of American elm was discovered in Afton, Minn., on an agricultural property, which was homesteaded in 1855 (U.S. granted title to Thomas Persons) before the tree came into existence.

### SUMMARY OF THE INVENTION

This new and distinct variety of American elm is typical of the species as locally represented in every apparent physical way, with the botanical description set forth below. This cultivar is, as typical of the species, vase-shaped, but in this case open-grown and spreading. Color and canopy density are excellent. The species is represented in USDA Hardiness zones 2 through 9 but is restricted to some extent by provenance, meaning seeds from American elm trees growing in Florida are not likely to prosper in Winnipeg, Manitoba. This cultivar is likely to be hardy in USDA hardiness zones 2 through 5 or 6. Given that this mature specimen has survived an unabated Dutch elm disease epidemic for over 30 years without visible injury or infection, while young, wild, American elm trees continue to become infected and die all around it, this cultivar is believed to be exceptionally tolerant to Dutch elm disease.

The tree was asexually reproduced by rooted cuttings and by grafting and budding onto established wild-type and U. americana ‘Valley Forge’ rootstocks. The asexual reproductions ran true to the originally discovered tree from root tip to shoot tip and to each other in all respects.

The asexual reproductions, along with wild-type U. americana, U. americana ‘Miller Park,’ U. americana ‘Valley Forge,’ and U. rubra elm trees, were inoculated with Ophiostoma novo-ulmi (about 0.5 ml of a solution containing ≧10⁶ spores/ml) by means of a hole about ⅛ inch in diameter drilled into the base of the trees, when 3-4 feet in height. The U. rubra and U. americana ‘Miller Park,’ ‘Valley Forge,’ and ‘St. Croix’ became symptomatic, but survived, whereas the wild-type elms wilted and died (R. A. Blanchette, unpublished data).

### BRIEF DESCRIPTION OF THE DRAWINGS

FIG. 1 is a photograph showing the timber form of ‘St. Croix.’

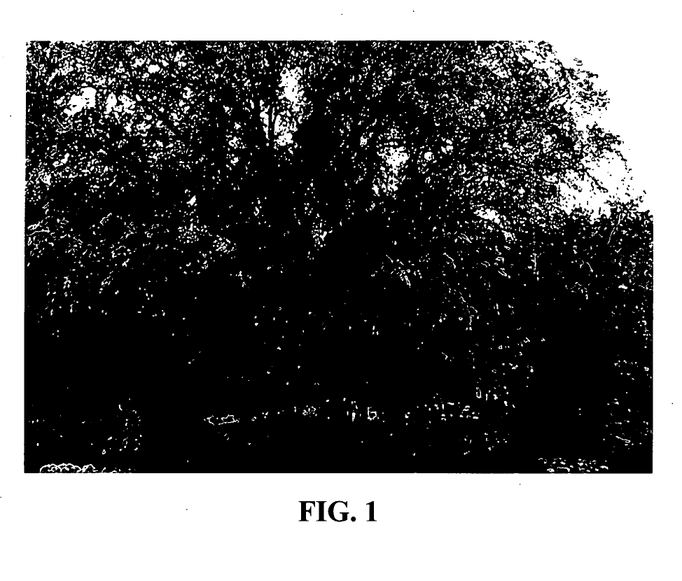

FIG. 2 is a photograph showing the timber form of ‘St Croix’ in the absence of leaves.

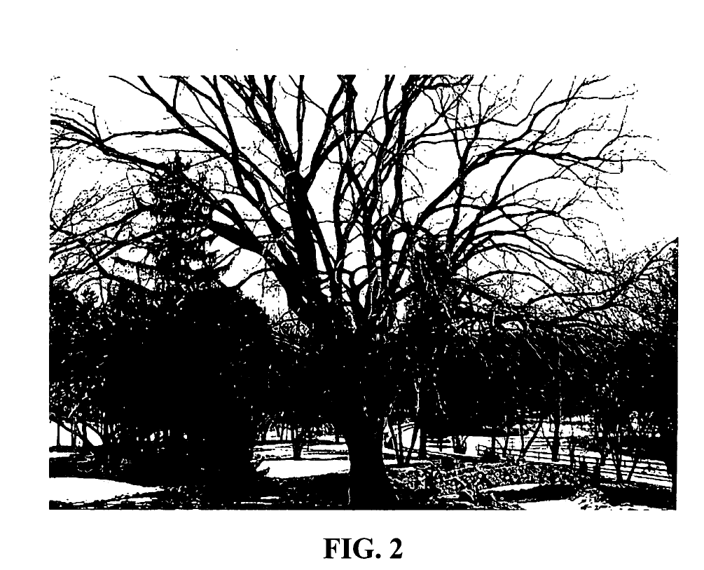

FIG. 3 is a photograph showing the bark of ‘St. Croix.’

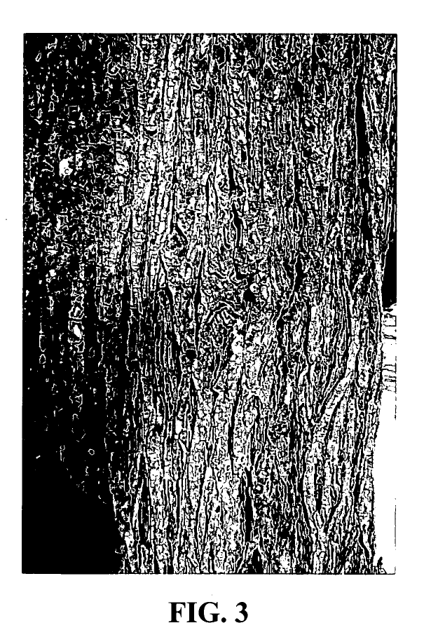

FIG. 4 is a photograph showing the twigs of ‘St. Croix.’

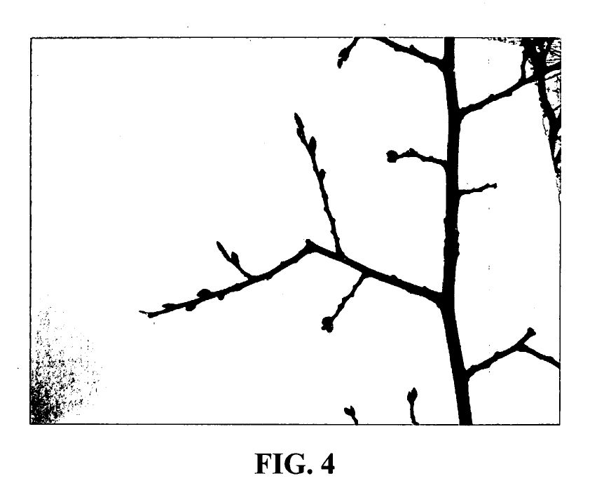

### DETAILED BOTANICAL DESCRIPTION OF THE PLANT

The botanical details of this new and distinct variety of American elm tree are as follows:

The ‘St. Croix’ is an exceptionally large specimen of the American elm species. The diameter at breast height (dbh; 4.5 feet above the ground) is about 75 inches (19 feet, 8 inches in circumference). It is about 75 feet high with a crown spread of about 110 feet. Its age is unknown, but likely to be between 80 and 110 years.

Vigor as a genetic measure of suitability for the site is excellent.

Hardiness on the USDA hardiness zone map is likely to be in zones 2 through 5 or 6.

General health and pest susceptibility: Vitality as a measure of health is very good, with good canopy density and excellent dark green color. The tree is normally susceptible to extant indigenous pests, all of minor importance.

Growth habit and rate: The ‘St. Croix’ has an open-grown, spreading, vase-shaped crown. The growth rate is fast and typical of the species in this part of Minnesota.

Bark is typically divided into grayish, flat-topped ridges, which are separated by roughly diamond-shaped fissures and which become indefinite in pattern in the canopy. Bark on young branches is smooth with inconspicuous lenticels.

Twigs are slender, zigzag, brown, glabrous or slightly pubescent; lateral buds are about ¼″ long, ovoid, acute but not sharp-pointed, smooth or sparingly downy, chestnut-brown.

Leaves are deciduous, simple, alternate, short-petioled, 2-ranked, dark green (closest to 006600 on HTML True Color Chart), 4 to 6 inches long, 1 to 3 inches wide and oblong-obovate to elliptical; margin coarsely doubly serrate; apex acuminate; base conspicuously inequilateral; surfaces glabrous (smooth) or slightly scabrous (roughened) above, usually pubescent below; veins alternate, ascending, parallel and extending from central vein to apex of longest serrations.

Flowers are vernal appearing before the leaves unfold, perfect, born in long-pedicelled fascicles of 3 or 4; ovary flattened, surmounted by a deeply 2-lobed style.

Fruit is a samara maturing in the spring as the leaves unfold; about ½ inch long, oval to oblong-obovate, deeply notched at apex, margin ciliate with smooth surfaces.

We claim:

1. A new and distinct variety of American elm tree named ‘St. Croix’ substantially as illustrated and described.
